(19) United States
(12) Patent Application Publication
(10) Pub. No.: US 2011/0287069 A1
(43) Pub. Date: Nov. 24, 2011

(54) SOLID ORAL FORMULATIONS OF A PYRIDOPYRIMIDINONE

(75) Inventors: Daya Verma, Edison, NJ (US); Yue (Helen) Teng, Morris Plains, NJ (US); Rajinder Singh, Sewaren, NJ (US); Dan Thompson, Dover, NJ (US)

(73) Assignee: Novartis AG, Basel (CH)

(21) Appl. No.: 13/146,678

(22) PCT Filed: Jan. 28, 2010
(86) PCT No.: PCT/US2010/022335

§ 371 Date: Jul. 28, 2011

Related U.S. Application Data
(60) Provisional application No. 61/148,160, filed on Jan. 29, 2009.

(51) Int. Cl. A61K 9/14; A61K 31/519; B82Y 5/00

(52) U.S. Cl. 424/400; 514/264.11; 977/773; 977/915

(57) ABSTRACT

A solid oral dosage pharmaceutical formulation of (R)-2-Amino-7-[4-fluoro-2-(6-methoxy-pyridin-2-yl)-phenyl]-4-methyl-7,8-dihydro-6H-pyrido[4,3-d]pyrimidin-5-one or its salt; and a surfactant or an acid.

### BACKGROUND OF THE INVENTION

1. Field of the Invention

This invention relates to solid oral formulations of (R)-2-amino-7-[4-fluoro-2-(6-methoxy-pyridin-2-yl)-phenyl]-4-methyl-7,8-dihydro-6H-pyrido[4,3-d]pyrimidin-5-one, as well as methods of treatment using the same.

2. Related Background Art

The compound (R)-2-amino-7-[4-fluoro-2-(6-methoxy-pyridin-2-yl)-phenyl]-4-methyl-7,8-dihydro-6H-pyrido[4,3-d]pyrimidin-5-one, has the formula (I):

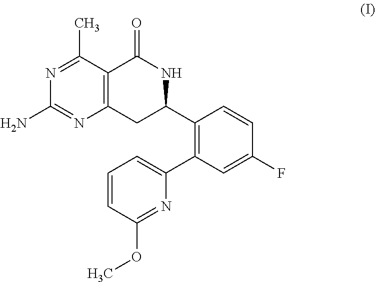

and is described in US Patent Application Publication 2007/0123546, which discloses important pharmacological properties of (R)-2-amino-7-[4-fluoro-2-(6-methoxy-pyridin-2-yl)-phenyl]-4-methyl-7,8-dihydro-6H-pyrido[4,3-d]pyrimidin-5-one, such as treating cancers and other disorders related to heat shock protein 90 (hsp 90).

### BRIEF SUMMARY OF THE INVENTION

The present invention is directed to oral formulations of (R)-2-amino-7-[4-fluoro-2-(6-methoxy-pyridin-2-yl)-phenyl]-4-methyl-7,8-dihydro-6H-pyrido[4,3-d]pyrimidin-5-one. Preferred embodiments of the present invention are directed to capsule and tablet formulations of (R)-2-amino-7-[4-fluoro-2-(6-methoxy-pyridin-2-yl)-phenyl]-4-methyl-7,8-dihydro-6H-pyrido[4,3-d]pyrimidin-5-one.

### BRIEF DESCRIPTION OF THE DRAWINGS

FIG. 1 is a dissolution profile (in pH 2 dissolution medium) of the present invention (triangle data points) in comparison to a formulation (square data points) without a small particle form of (R)-2-amino-7-[4-fluoro-2-(6-methoxy-pyridin-2-yl)-phenyl]-4-methyl-7,8-dihydro-6H-pyrido[4,3-d]pyrimidin-5-one, and without a surfactant or an acid.

### DETAILED DESCRIPTION OF THE INVENTION

(R)-2-Amino-7-[4-fluoro-2-(6-methoxy-pyridin-2-yl)-phenyl]-4-methyl-7,8-dihydro-6H-pyrido[4,3-d]pyrimidin-5-one is a compound with a very low solubility. In an aqueous media with a pH above 3, (R)-2-amino-7-[4-fluoro-2-(6-methoxy-pyridin-2-yl)-phenyl]-4-methyl-7,8-dihydro-6H-pyrido[4,3-d]pyrimidin-5-one has very low solubility. Even at a lower pH of 1, the dissolution rate of (R)-2-amino-7-[4-fluoro-2-(6-methoxy-pyridin-2-yl)-phenyl]-4-methyl-7,8-dihydro-6H-pyrido[4,3-d]pyrimidin-5-one is too slow. The present inventors have discovered that, when (R)-2-amino-7-[4-fluoro-2-(6-methoxy-pyridin-2-yl)-phenyl]-4-methyl-7,8-dihydro-6H-pyrido[4,3-d]pyrimidin-5-one is in the presence of a surfactant or an acid, the problems of a low solubility and a slow dissolution rate are overcome, resulting in (R)-2-amino-7-[4-fluoro-2-(6-methoxy-pyridin-2-yl)-phenyl]-4-methyl-7,8-dihydro-6H-pyrido[4,3-d]pyrimidin-5-one being soluble in aqueous solution and a dissolution rate that is unexpectedly faster and higher.

By increasing solubility and the dissolution rate, therefore, the dosage forms of the present invention may enhance the bioavailability of (R)-2-amino-7-[4-fluoro-2-(6-methoxy-pyridin-2-yl)-phenyl]-4-methyl-7,8-dihydro-6H-pyrido[4,3-d]pyrimidin-5-one and lessen undesirable characteristics of administration of a poorly soluble active agent, such as the food effect, as well as increase patient compliance. The formulations of the present invention have also been found to be stable upon room temperature storage.

Small particle (R)-2-amino-7-[4-fluoro-2-(6-methoxy-pyridin-2-yl)-phenyl]-4-methyl-7,8-dihydro-6H-pyrido[4,3-d]pyrimidin-5-one is preferably present in a micronized form or a nano form, having a median particle size of about 10 nm to about 40 microns. When present in a micronized form, effective median particle size ranges include about 0.5 to about 40 microns, about 0.5 to about 20 microns, about 0.5 to about 20 microns, preferably about 0.5 to about 5 microns, more preferably about 1 to about 4 microns. Micronization can be achieved by any known method, such as grinding and milling using standard equipment such as a fluid energy mill or a jet mill.

When present in a nano form, effective small particle ranges include about 5 to about 1000 nanometers, about 10 to about 100 nanometers, and about 10 to about 50 nanometers. Nano sized small particle forms can be formed by conventional means with conventional equipment, such as nanomills, including nanomills with beads or by spray drying the nano-sized active ingredient onto an excipient, such an microcrystalline cellulose. Nano-sized active ingredient could also be obtained by spry drying the active with solubilizing excipients, which could be a surfactant and or acidifier, or a solubility enhancing excipients which may be a polymer, lipidic excipient, oils.

The small particle and non-small particle forms of (R)-2-amino-7-[4-fluoro-2-(6-methoxy-pyridin-2-yl)-phenyl]-4-methyl-7,8-dihydro-6H-pyrido[4,3-d]pyrimidin-5-one can be present in crystalline or amorphous form, or mixtures thereof. Salt forms of (R)-2-amino-7-[4-fluoro-2-(6-methoxy-pyridin-2-yl)-phenyl]-4-methyl-7,8-dihydro-6H-pyrido[4,3-d]pyrimidin-5-one include HCl, tosic, methanesulfonic, benzenesulfonic, oxalic, ethanesulfonic, aspartic, maleic, and H₂SO₄.

As used herein, the term “pharmaceutically acceptable salts” refers to the nontoxic acid or alkaline earth metal salts of (R)-2-amino-7-[4-fluoro-2-(6-methoxy-pyridin-2-yl)-phenyl]-4-methyl-7,8-dihydro-6H-pyrido[4,3-d]pyrimidin-5-one of the invention. These salts can be prepared in situ during the final isolation and purification of (R)-2-amino-7-[4-fluoro-2-(6-methoxy-pyridin-2-yl)-phenyl]-4-methyl-7,8-dihydro-6H-pyrido[4,3-d]pyrimidin-5-one, or by separately reacting the base or acid functions with a suitable organic or inorganic acid or base, respectively. Representative salts include, but are not limited to, the following: acetate, adipate, alginate, citrate, aspartate, benzoate, benzenesulfonate, a bile salt, bisulfate, butyrate, camphorate, camphorsulfonate, digluconate, cyclopentanepropionate, dodecylsulfate, ethanesulfonate, glucoheptanoate, glycerophosphate, hemi-sulfate, heptanoate, hexanoate, fumarate, hydrochloride, hydrobromide, hydroiodide, 2-hydroxyethanesulfonate, lactate, maleate, methanesulfonate, nicotinate, 2-napthalenesulfonate, oxalate, pamoate, pectinate, persulfate, 3-phenylproionate, picrate, pivalate, propionate, succinate, sulfate, tartrate, thiocyanate, p-toluenesulfonate, and undecanoate. Also, the basic nitrogen-containing groups can be quaternized with such agents as alkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl, and stearyl chlorides, bromides and iodides, aralkyl halides like benzyl and phenethyl bromides, and others. Water or oil-soluble or dispersible products are thereby obtained.

Examples of acids that may be employed to form pharmaceutically acceptable acid addition salts include such inorganic acids as hydrochloric acid, sulfuric acid and phosphoric acid and such organic acids as oxalic acid, maleic acid, methanesulfonic acid, succinic acid and citric acid. Basic addition salts can be prepared in situ during the final isolation and purification of (R)-2-amino-7-[4-fluoro-2-(6-methoxy-pyridin-2-yl)-phenyl]-4-methyl-7,8-dihydro-6H-pyrido[4,3-d]pyrimidin-5-one, or separately by reacting carboxylic acid moieties with a suitable base such as the hydroxide, carbonate or bicarbonate of a pharmaceutically acceptable metal cation or with ammonia, or an organic primary, secondary or tertiary amine. Pharmaceutically acceptable salts include, but are not limited to, cations based on the alkali and alkaline earth metals, such as sodium, lithium, potassium, calcium, magnesium, aluminum salts and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Other representative organic amines useful for the formation of base addition salts include diethylamine, ethylenediamine, ethanolamine, diethanolamine, piperazine, and the like.

The formulation according to the invention may contain pharmaceutically acceptable excipients commonly used in pharmaceutical formulations, particularly those for oral administration.

In a preferred embodiment according to the invention the formulation may be in the form of an oral solid dosage formulation comprising (R)-2-amino-7-[4-fluoro-2-(6-methoxy-pyridin-2-yl)-phenyl]-4-methyl-7,8-dihydro-6H-pyrido[4,3-d]pyrimidin-5-one or a salt thereof, and a surfactant, or an acid; or both a surfactant and an acid, with optionally one or more additional excipients. Examples of additional excipients include a disintegrant or super disintegrant, a filler, a glidant, or a lubricant. The (R)-2-amino-7-[4-fluoro-2-(6-methoxy-pyridin-2-yl)-phenyl]-4-methyl-7,8-dihydro-6H-pyrido[4,3-d]pyrimidin-5-one can be in small particle form

Surfactants suitable for the present invention include vitamin E TPGS, polysorbate 80, polysorbate 20, sodium lauryl sulfate, anionic surfactants of the alkyl sulfate type, for example sodium, potassium or magnesium n-dodecyl sulfate, n-tetradecyl sulfate, n-hexadecyl sulfate or n-octadecyl sulfate, of the alkyl ether sulfate type, for example sodium, potassium or magnesium n-dodecyloxyethyl sulfate, n-tetradecyloxyethyl sulfate, n-hexadecyloxyethyl sulfate or n-octadecyloxyethyl sulfate, or of the alkanesulfonate type, for example sodium, potassium or magnesium n-dodecanesulfonate, n-tetradecanesulfonate, n-hexadecanesulfonate or n-octadecanesulfonate, or non-ionic surfactants of the fatty acid polyhydroxy alcohol ester type, such as sorbitan monolaurate, monooleate, monostearate or monopalmitate, sorbitan tristearate or trioleate, polyoxyethylene adducts of fatty acid polyhydroxy alcohol esters, such as polyoxyethylene sorbitan monolaurate, monooleate, monostearate, monopalmitate, tristearate or trioleate, polyethylene glycol fatty acid esters, such as polyoxyethyl stearate, polyethylene glycol 400 stearate, polyethylene glycol 2000 stearate, especially ethylene oxide/propylene oxide block polymers of the PLURONICS (BWC) or SYNPERONIC (ICI) type.

Vitamin E TPGS (d-alpha tocopheryl polyethylene glycol 1000 succinate) is normally a waxy substance at room temperature, which is difficult to process; however it can made into a particulate form by freezing and then milling, which allows for direct blending of the vitamin E TPGS. A direct blending process is one that involves the dry processing of an excipient such as vitamin E TPGS and the active ingredient, in this case (R)-2-amino-7-[4-fluoro-2-(6-methoxy-pyridin-2-yl)-phenyl]-4-methyl-7,8-dihydro-6H-pyrido[4,3-d]pyrimidin-5-one. Dry processing means that the excipients are processed in a dry state and not melted, and moreover do not form a solid solution or solid dispersion. Vitamin E TPGS can be direct blended made by freezing and milling can be processed more easily, and can be present in the composition in an amounts up to about 20%, about 25%, or about 35%, or about 40%, or less than 50% (w/w). Dry processed vitamin E TPGS is present in the present invention in a powered or particulate form.

Surfactants for the present invention can be present in the formulation as about 0.5% to about 95%, about 1% to about 85%, and about 5% to about 75% (w/w) of the composition. In addition, compositions having about 5%, about 10%, about 15%, about 20%, about 25%, about 30%, about 35% and about 45% surfactant are envisioned.

Acids for use with the present invention include any pharmaceutically acceptable acid, including organic acids such as succinic acid, tartaric acid, citric acid, acetic acid, propionic acid, maleic acid, malic acid, phthalic acid, methanesulfonic acid, toluenesulfonic acid, napthalenesulfonic acid, camphorsulfonic acid, benzenesulfonic acid, lactic acid, butyric acid, hydroxymaleic acid, malonic acid, sorbic acid, glycolic acid, glucoronic acid, fumaric acid, mucic acid, gluconic acid, benzoic acid, oxalic acid, phenylacetic acid, salicyclic acid, sulphanilic acid, aspartic acid, glutamic acid, edetic acid, stearic acid, palmitic acid, oleic acid, lauric acid, pantothenic acid, tannic acid, valeric acid or ascorbic acid, and a polymeric acid such as methacrylic acid copolymer, EUDRAGIT E PO, EUDRAGIT L100-55, EUDRAGIT L-30 D-55, EUDRAGIT FS 30 D, EUDRAGIT NE 30 D, EUDRAGIT L100, EUDRAGIT S100, a poly-amino acid (e.g., poly-glutamic acid, poly-aspartic acid and combinations thereof), poly-nucleic acids, poly-acrylic acid, poly-galacturonic acid, and poly-vinyl sulfate or an anionic amino acid, such as polymer poly-glutamic acid or poly-aspartic acid. For purposes of describing the present invention, organic acids are understood to include polymeric acids. Acids can also include inorganic acids such as hydrochloric acid, phosphoric acid, phosphonic acid, phosphinic acid, boronic acid, hydrobromic acid, sulfuric acid, sulfamic acid, nitric acid, or sulfonic acid. The acid can be present as a buffer.

Acids for the present invention can be present in the formulation as about 2% to about 80%, about 2% to about 60%, and about 5% to about 40% (w/w) of the composition. In addition, compositions having about 10%, about 20%, about 25%, about 35%, about 40%, and about 45% acid are envisioned.

Disintegrants for use with the present invention can include traditional disintegrants, such as starch, alginic acid or amberlite resins; also included are super disintegrants, such as crospovidone, sodium starch glycolate, croscarmellose sodium, and soy polysaccharide. The term “super disintegrant” is a term well known in the art and denotes a disintegrant that is effective in lower concentrations in comparison to starch, generally at 2 to 4% w/w.

Glidants for use with the present invention include silicon dioxide, such as colloidal silicon dioxide (fumed silica) and talc.

An example of a lubricant that can be used with the present invention is magnesium stearate, stearic acid, talc, hydrogenated vegetable oil, gylceryl behenete, sodium stearyl fumarate, PEG 4000/6000, sodium lauryl sulphate, isoleucine, sodium benzoate, or fumed silica.

Fillers can be used with the present invention, such as talcum, silicon dioxide, for example synthetic amorphous anhydrous silicic acid of the SYLOID type (Grace), for example SYLOID 244 FP, microcrystalline cellulose (MCC), for example of the AVICEL type (FMC Corp.), for example of the types AVICEL PH101, 102, 105, RC581 or RC 591, EMCOCEL type (Mendell Corp.) or ELCEMA type (Degussa); carbohydrates, such as sugars, sugar alcohols, starches or starch derivatives, for example sucrose, lactose, dextrose, saccharose, glucose, sorbitol, mannitol, xylitol, potato starch, maize starch, rice starch, wheat starch or amylopectin, tricalcium phosphate, calcium hydrogen phosphate, calcium sulfate, dibasic calcium phosphates, or magnesium trisilicate.

Suitable binders that can be used with the present invention include gelatin, tragacanth, agar, alginic acid, cellulose ethers, for example methylcellulose, carboxymethylcellulose or hydroxypropylmethylcellulose, polyethylene glycols or ethylene oxide homopolymers, especially having a degree of polymerization of approximately from 2.0×10³ to 1.0×10⁵ and an approximate molecular weight of about from 1.0×10⁵ to 5.0×10⁶, for example excipients known by the name POLYOX (Union Carbide), polyvinylpyrrolidone or povidones, especially having a mean molecular weight of approximately 1000 and a degree of polymerization of approximately from 500 to 2500, and also agar or gelatin.

The formulation of the present invention can be manufactured with a standard process, such as direct blending, direct compression, granulation, solvent granulation, wet granulation, fluid-bed granulation, (hot) melt granulation, dry granulation, roller compaction, slugging, freeze dried tabletting, wet or dry aggregation, and extrusion and spheronization.

In one embodiment, the present invention is formulated as a capsule, such as hard gelatin capsule or a soft elastic capsule. Alternatively, the present invention is in the form of a tablet or a pill. In these solid oral formulations the amount of (R)-2-amino-7-[4-fluoro-2-(6-methoxy-pyridin-2-yl)-phenyl]-4-methyl-7,8-dihydro-6H-pyrido[4,3-d]pyrimidin-5-one can be present in the ranges of 1-500 mg, 2.5-250 mg, or 2.5-100 mg, with preferred examples including 1 mg, 2.5 mg, 5 mg, 10 mg, 20 mg, 25 mg, 50 mg, 100 mg, and 200 mg.

The solid oral formulations of the present invention can be administered to treat diseases related to the inhibition of hsp 90, including cancer and cancer tumors, such as breast, ovarian, prostate, chronic myelogenous leukemia (CML), melanoma, gastrointestinal stromal tumors (GISTs), master cell leukemia, testicular tumor, acute myelogenous leukemia, gastric tumor, lung, head, neck, glioblastoma, colon, thyroid, stomach, liver, multiple myeloma, renal, and lymphoma.

The exact dosage regimen of (R)-2-amino-7-[4-fluoro-2-(6-methoxy-pyridin-2-yl)-phenyl]-4-methyl-7,8-dihydro-6H-pyrido[4,3-d]pyrimidin-5-one in the formulations of the present invention can be determined by one of skill in art upon consideration of the condition and requirements of the patient. For example, the present invention could be administered daily, every other day or weekly.

The following Examples illustrate the invention.

### Example 1

The below Table 1 illustrates capsules with 2.5 mg and 20 mg of (R)-2-amino-7-[4-fluoro-2-(6-methoxy-pyridin-2-yl)-phenyl]-4-methyl-7,8-dihydro-6H-pyrido[4,3-d]pyrimidin-5-one.

TABLE 1

| | 2.5/10 mg | 20/50 mg |
|---|---|---|
| | Active Agent | Active Agent |
| Ingredient | (% w/w) | (% w/w) |
| Micronized (R)-2-amino-7-[4- | 2.5 | 20.0 |
| fluoro-2-(6-methoxy-pyridin-2- | | |
| yl)-phenyl]-4-methyl-7,8- | | |
| dihydro-6H-pyrido[4,3- | | |
| d]pyrimidin-5-one (active agent) | | |
| Microcrystalline cellulose | 60.3 | 42.8 |
| (AVICEL PH 101) | | |
| Vitamin E TPGS | 10.0 | 10.0 |
| Succinic acid | 20.0 | 20.0 |
| Crospovidone | 6.0 | 6.0 |
| Fumed silica | 0.4 | 0.4 |
| AEROSIL | | |
| Mg Stearate | 0.8 | 0.8 |
| Total | 100.0 | 100.0 |

(R)-2-Amino-7-[4-fluoro-2-(6-methoxy-pyridin-2-yl)-phenyl]-4-methyl-7,8-dihydro-6H-pyrido[4,3-d]pyrimidin-5-one was micronized and screened through a #25 screen to a D average particle size of about 2 microns, with laser light diffraction yielding the following results: D(10) 0.78 μm, D(50) 2.18 μm, and D(90) 3.95 μm. The micronized (R)-2-amino-7-[4-fluoro-2-(6-methoxy-pyridin-2-yl)-phenyl]-4-methyl-7,8-dihydro-6H-pyrido[4,3-d]pyrimidin-5-one was then screened and added to a mixing bin along with the succinic acid and part A (50%) of the microcrystalline cellulose. The contents in the mixing bin were mixed for 150 revolutions, screened through a #40 screen and combine with a screened (#40 screen) mixture of AEROSIL 200, crospovidone, and the remaining other part B (50%) of the microcrystalline cellulose. This combination was mixed for 250 revolutions and screened through a #40 screen, and then combine with a first frozen and then milled mixture of vitamin E TPGS (screen no. 0063 using a Fitz mill) and magnesium stearate that was passed through at #30 mesh, to form a final combination, which was then blended together for 150 revolutions and encapsulated in a hard gelatin capsule using an encapsulation machine.

### Example 2

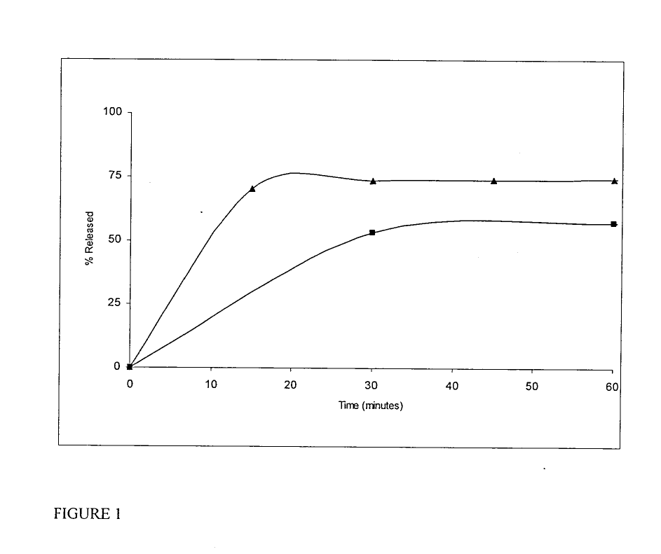

A 50 mg preparation of micronized (R)-2-amino-7-[4-fluoro-2-(6-methoxy-pyridin-2-yl)-phenyl]-4-methyl-7,8-dihydro-6H-pyrido[4,3-d]pyrimidin-5-one was prepared in accordance with Table 1 above and then assayed in reverse phase HPLC to determine the percentage of dissolved active agent. Gradient chromatographic conditions were used. Mobile phase A was 90% 0.01M ammonium phosphate in water, pH 6.3, buffered with phosphoric acid, and 10% acetonitrile. Mobile phase B was 100% acetonitrile. 10 micro liters of assay solution was injected. Run time was five minutes, column temperate was 40° C., and the detection wavelength was 268 nm. Results are shown in FIG. 1 as the triangle-shaped data points. Within 10 minutes, more the 50% of the (R)-2-amino-7-[4-fluoro-2-(6-methoxy-pyridin-2-yl)-phenyl]-4-methyl-7,8-dihydro-6H-pyrido[4,3-d]pyrimidin-5-one was dissolved.

### Example 3

A formulation based on Example 1 was administered in dogs The AUC value (hour*ng/mL) was 7420 for fasted dogs, with a Tmax of 1.8. This compared favorably to administration of a 0.5% methylcellulose suspension which had an AUC of 3760 and 2 Tmax for fasted dogs and 10400 AUC with a 4 Tmax for fed dogs.

### Example 4

A formulation based on Example 1 was administered to human patients in 2.5 mg and 5 mg concentrations. No toxic effects from the formulation were observed. The dose showed a desirable linear correlation between plasma concentration and time. The peak plasma concentration occurred in 3 hours. The half time for elimination from the body (T½) was between 14.8 to 45.3 hours.

### Comparative Example 1

TABLE 2

| | 50 mg |
|---|---|
| | Active Agent |
| Ingredient | (% w/w) |
| (R)-2-Amino-7-[4-fluoro-2-(6- | 20.0 |
| methoxy-pyridin-2-yl)-phenyl]- | |
| 4-methyl-7,8-dihydro-6H- | |
| pyrido[4,3-d]pyrimidin-5-one | |
| (active agent) | |
| Microcrystalline cellulose | 72.5 |
| Povidone (PVP K30) | 3.2 |
| Crospovidone | 3.2 |
| Fumed silica | 0.3 |
| AEROSIL | |
| Mg Stearate | 0.8 |
| Total | 100.0 |

In accordance with the weight percentages of Table 2 above, (R)-2-amino-7-[4-fluoro-2-(6-methoxy-pyridin-2-yl)-phenyl]-4-methyl-7,8-dihydro-6H-pyrido[4,3-d]pyrimidin-5-one, microcrystalline cellulose, povidone, and crospovidone were blended together in a mixer and then ground in a pestle mortar to insure uniformity. The magnesium stearate and AEROSIL were then added to the mixture and blended for ten minutes. A sample was then assayed in accordance with above Example 2. 50% of active was not dissolved until approximately 30 minutes after exposure in solution.

1. A pharmaceutical formulation comprising
(a) (R)-2-amino-7-[4-fluoro-2-(6-methoxy-pyridin-2-yl)-phenyl]-4-methyl-7,8-dihydro-6H-pyrido[4,3-d]pyrimidin-5-one or a pharmaceutically acceptable salt thereof; and at least one
(b) a surfactant,
(c) or an acid,
wherein the pharmaceutical formulation is a solid oral dosage form, and the (R)-2-amino-7-[4-fluoro-2-(6-methoxy-pyridin-2-yl)-phenyl]-4-methyl-7,8-dihydro-6H-pyrido[4,3-d]pyrimidin-5-one or its salt can be in small particle form with a median particle size of about 10 nm to about 40 microns,
with the proviso that if the surfactant is vitamin E TPGS, it is processed by direct blending.

2. The pharmaceutical formulation of claim 1, wherein the surfactant is vitamin E TPGS, polysorbate 80, polysorbate 20, sodium lauryl sulfate, an alkyl sulfate anionic surfactant, sodium, potassium n-dodecyl sulfate magnesium n-dodecyl sulfate, n-tetradecyl sulfate, n-hexadecyl sulfate, n-octadecyl sulfate, an alkyl ether sulfate, sodium n-dodecyloxyethyl sulfate, potassium n-dodecyloxyethyl sulfate, magnesium n-dodecyloxyethyl sulfate, n-tetradecyloxyethyl sulfate, n-hexadecyloxyethyl sulfate, n-octadecyloxyethyl sulfate, an alkanesulfonate, sodium n-dodecanesulfonate, potassium n-dodecanesulfonate, magnesium n-dodecanesulfonate, n-tetradecanesulfonate, n-hexadecanesulfonate, n-octadecanesulfonate, a non-ionic fatty acid polyhydroxy alcohol ester surfactant, sorbitan monolaurate, sorbitan monooleate, sorbitan monostearate, sorbitan monopalmitate, sorbitan tristearate, sorbitan trioleate, a polyoxyethylene fatty acid, a polyhydroxy alcohol ester, polyoxyethylene sorbitan monolaurate, polyoxyethylene sorbitan monooleate, polyoxyethylene sorbitan monostearate, polyoxyethylene sorbitan monopalmitate, polyoxyethylene sorbitan tristearate, polyoxyethylene sorbitan trioleate, a polyethylene glycol fatty acid ester, polyoxyethyl stearate, polyethylene glycol 400 stearate, polyethylene glycol 2000 stearate, an ethylene oxide block polymer, or a propylene oxide block polymer.

3. The pharmaceutical formulation of claim 2, wherein the surfactant is vitamin E TPGS, polysorbate 80 or sodium lauryl sulfate.

4. The composition of claim 3, wherein the surfactant is vitamin E TPGS.

5. The pharmaceutical formulation of claim 1, wherein the acid is an organic acid.

6. The pharmaceutical formulation of claim 5, wherein the organic acid is succinic acid, tartaric acid, citric acid, acetic acid, propionic acid, citric acid, maleic acid, malic acid, phthalic acid, methanesulfonic acid, toluenesulfonic acid, napthalenesulfonic acid, camphorsulfonic acid, benzenesulfonic acid, lactic acid, butyric acid, hydroxymaleic acid, malonic acid, sorbic acid, glycolic acid, glucoronic acid, fumaric acid, mucic acid, gluconic acid, benzoic acid, oxalic acid, phenylacetic acid, salicyclic acid, sulphanilic acid, aspartic acid, glutamic acid, edetic acid, stearic acid, palmitic acid, oleic acid, lauric acid, pantothenic acid, tannic acid, valeric acid, ascorbic acid, a polymeric acid, methacrylic acid copolymer, poly-amino acid, poly-nucleic acids, poly-acrylic acid, poly-galacturonic acid, poly-vinyl sulfate, an anionic amino acid, a polymer poly-glutamic acid or a poly-aspartic acid.

7. The pharmaceutical formulation of claim 6, wherein the organic acid is succinic acid.

8. A pharmaceutical formulation comprising
(a) (R)-2-amino-7-[4-fluoro-2-(6-methoxy-pyridin-2-yl)-phenyl]-4-methyl-7,8-dihydro-6H-pyrido[4,3-d]pyrimidin-5-one or a pharmaceutically acceptable salt thereof;
(b) a surfactant and
(c) an acid,
wherein the pharmaceutical formulation is a solid oral dosage form, and the (R)-2-amino-7-[4-fluoro-2-(6-methoxy-pyridin-2-yl)-phenyl]-4-methyl-7,8-dihydro-6H-pyrido[4,3-d]pyrimidin-5-one or its salt can be in small particle form with a median particle size of about 10 nm to about 40 microns,
with the proviso that if the surfactant is vitamin E TPGS, it is processed by direct blending.

9. The pharmaceutical formulation of claim 8, wherein the surfactant is vitamin E TPGS, polysorbate 80, polysorbate 20, sodium lauryl sulfate, an alkyl sulfate anionic surfactant, sodium, potassium n-dodecyl sulfate magnesium n-dodecyl sulfate, n-tetradecyl sulfate, n-hexadecyl sulfate, n-octadecyl sulfate, an alkyl ether sulfate, sodium n-dodecyloxyethyl sulfate, potassium n-dodecyloxyethyl sulfate, magnesium n-dodecyloxyethyl sulfate, n-tetradecyloxyethyl sulfate, n-hexadecyloxyethyl sulfate, n-octadecyloxyethyl sulfate, an alkanesulfonate, sodium n-dodecanesulfonate, potassium n-dodecanesulfonate, magnesium n-dodecanesulfonate, n-tetradecanesulfonate, n-hexadecanesulfonate, n-octadecanesulfonate, a non-ionic fatty acid polyhydroxy alcohol ester surfactant, sorbitan monolaurate, sorbitan monooleate, sorbitan monostearate, sorbitan monopalmitate, sorbitan tristearate, sorbitan trioleate, a polyoxyethylene fatty acid, a polyhydroxy alcohol ester, polyoxyethylene sorbitan monolaurate, polyoxyethylene sorbitan monooleate, polyoxyethylene sorbitan monostearate, polyoxyethylene sorbitan monopalmitate, polyoxyethylene sorbitan tristearate, polyoxyethylene sorbitan trioleate, a polyethylene glycol fatty acid ester, polyoxyethyl stearate, polyethylene glycol 400 stearate, polyethylene glycol 2000 stearate, an ethylene oxide block polymer, or a propylene oxide block polymer.

10. The composition of claim 9, wherein the surfactant is vitamin E TPGS.

11. A pharmaceutical formulation of claim 10, wherein the surfactant is vitamin E TPGS and the median particle size of the small particle form of (R)-2-amino-7-[4-fluoro-2-(6-methoxy-pyridin-2-yl)-phenyl]-4-methyl-7,8-dihydro-6H-pyrido[4,3-d]pyrimidin-5-one or its salt is about 10 nm to about 40 microns.

12. The pharmaceutical formulation of claim 11, wherein the median particle size of (R)-2-amino-7-[4-fluoro-2-(6-methoxy-pyridin-2-yl)-phenyl]-4-methyl-7,8-dihydro-6H-pyrido[4,3-d]pyrimidin-5-one or its salt is about 0.5 to about 40 microns and further comprising a super disintegrant.

13. The pharmaceutical formulation of claim 1, further comprising a disintegrant or super disintegrant.

14. A pharmaceutical formulation comprising
(a) a micronized pharmaceutical agent,
(b) vitamin E TPGS, and
(c) an organic acid.

15. The pharmaceutical formulation of claim 14, wherein the organic acid is succinic acid.

16. The pharmaceutical formulation of claim 8, further comprising a disintegrant or super disintegrant.
